(19) United States
(12) Patent Application Publication
(10) Pub. No.: US 2024/0301335 A1
(43) Pub. Date: Sep. 12, 2024

(54) PHOTOBIOREACTOR SYSTEMS AND METHODS

(71) Applicant: Lillianah Technologies Inc., Spring, TX (US)

(72) Inventors: Benjamin Sidney Slotnick, Spring, TX (US); Tarynn Daye Brewer, Spring, TX (US)

(21) Appl. No.: 18/180,702

(22) Filed: Mar. 8, 2023

(51) Int. Cl. C12M 1/00; A01G 33/00; C12M 1/34; C12M 1/36; C12N 1/12

(52) U.S. Cl. CPC C12M 21/02; A01G 33/00; C12M 31/10; C12M 41/06; C12M 41/36; C12M 41/44; C12M 41/48; C12N 1/12

(57) ABSTRACT

A photobioreactor system includes a frame, a reflective wall coupled to the frame such that the reflective wall defines and circumferentially surrounds an interior chamber, and multiple light sources coupled to the reflective wall such that the multiple light sources circumferentially surround the interior chamber. The interior chamber is configured to receive a container that holds and supports growth of a desired organism.

### FIELD OF DISCLOSURE

The present disclosure relates generally to photobioreactor systems and methods. More specifically, embodiments of the present disclosure relate to photobioreactor systems and methods that may be utilized to facilitate growth of desired organisms for any of a variety of purposes, such as for distribution into volumes of water (e.g., aquatic environments) and/or to provide nutrient supplements (e.g., to farm animals, such as cattle).

### BACKGROUND

The oceans are vast bodies of water covering 70 percent of the surface of Earth. A wide variety of marine life including plants and animals live throughout the oceans. Many parts of the oceans have the potential to harbor more, or different, types of life than they currently do. In these areas, quantity of living organisms, total biomass, and/or biodiversity is limited by low concentrations or a complete absence of specific nutrients and/or materials. Many approaches have been proposed and/or attempted to alleviate these conditions, either temporarily or permanently, but these approaches are not entirely effective or are prohibitively expensive.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In certain embodiments, a photobioreactor system includes a frame, a reflective wall coupled to the frame such that the reflective wall defines and circumferentially surrounds an interior chamber, and multiple light sources coupled to the reflective wall such that the multiple light sources circumferentially surround the interior chamber. The interior chamber is configured to receive a container that holds and supports growth of a desired organism.

In addition, in certain embodiments, a photobioreactor system configured to be positioned proximate to a water surface of a body of water includes a floating structure and a container that forms the floating structure or that is supported on the floating structure. The container is configured to hold contents that comprise a desired organism. The photobioreactor system also includes multiple light sources configured to emit light into the container and an air system with a pump and at least one tubing assembly, wherein the at least one tubing assembly includes at least one tubing that extends from the pump to at least one air distribution device that is configured to be placed in the container.

In addition, in certain embodiments, an environmental treatment method includes placing a desired organism, nutrients, and/or water into a container. The method also includes placing an air outlet of an air distribution system into the container, providing a light source to emit light into the container, monitoring growth of the desired organism within the container, transferring the container to a distribution site in an aquatic environment, and distributing contents of the container into the aquatic environment.

Various refinements of the features noted above may be undertaken in relation to various aspects of the present disclosure. Further features may also be incorporated in these various aspects as well. These refinements and additional features may exist individually or in any combination.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a perspective view of a photobioreactor system, in accordance with embodiments the present disclosure;

FIG. 2 is a cross-sectional side view of the photobioreactor system of FIG. 1, in accordance with embodiments the present disclosure;

FIG. 3 is a perspective view of a photobioreactor system with a frame assembly that supports multiple containers, in accordance with embodiments the present disclosure;

FIG. 4 is a perspective view of a photobioreactor system with a light source positioned within a container, in accordance with embodiments the present disclosure; and

FIG. 5 is a perspective view of a photobioreactor system located in an aquatic environment, in accordance with embodiments the present disclosure.

### DETAILED DESCRIPTION

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure. Further, to the extent that certain terms such as parallel, perpendicular, and so forth are used herein, it should be understood that these terms allow for certain deviations from a strict mathematical definition, for example to allow for deviations associated with manufacturing imperfections and associated tolerances.

When introducing elements of various embodiments of the present disclosure, the articles “a,”“an,” and “the” are intended to mean that there are one or more of the elements. The terms “comprising,”“including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

Provided herein are photobioreactor systems and methods to facilitate growth of desired organisms. For example, the photobioreactor systems and methods may be utilized to facilitate growth of microalgae. Further, the photobioreactor may be utilized to facilitate the growth of the desired organisms for any of a variety of purposes, including for distribution into volumes of water. In such cases, the photobioreactor system may be utilized in conjunction with and/or as part of an environmental treatment distribution system that promotes intentional change of conditions of an aquatic environment (e.g., coastal marine, nearshore marine, open ocean, deep ocean, lakes, ponds, rivers, streams, bays, wetlands, and/or estuaries) via controlled distribution of the desired organisms. Introduction of the desired organisms may be entirely or partially intended to induce an effect, condition, change, lack of change, or stasis in the aquatic environment and/or in a related environment impacted by the aquatic environment. For example, the environmental treatment distribution system may operate to decrease aquatic hypoxia, increase aquatic oxygenation, increase export of materials to depth, enhance fisheries, treat harmful algal bloom, address red tide, address nutrient runoff, address climate change, address eutrophication, and/or address environmental changes associated with commercial presence. The environmental treatment distribution system may operate to create conditions favorable to biologic growth (e.g., create an algal bloom). The environmental treatment distribution system may operate to create conditions favorable to capture of carbon dioxide (CO₂) and/or or other carbon-based materials, elements, and/or molecules, with or without the intent to sequester said carbon for some period of time. It should be appreciated that the photobioreactor systems and methods may be utilized to facilitate growth of the desired organisms for consumption by humans and/or other animals (e.g., cattle). It should also be appreciated that the photobioreactor systems and methods may be added in parallel with (e.g., used in conjunction with) wastewater treatment facilities to help clean water.

The photobioreactor system may grow the desired organisms in presence of nutrients. By way of example, the nutrients may include, but are not limited to, silica, iron, cobalt, copper, aluminum, nitrogen, phosphorous, magnesium, manganese, calcium, sodium, potassium, and carbon, all in various forms and solutions. In some embodiments, the nutrients may include volcanic ash or synthetic volcanic ash (e.g., volcanic ash in combination with one or more other nutrients or volcanic ash alone without other nutrients). The nutrients may or may not be in elemental form (e.g., may or may not be part of a more complex molecule). Further, the nutrients may be chosen due to their characteristics including but not limited to how they dissolve in different bodies and/or composition of water. The nutrients may be chosen based on visible characteristics including but not limited to grain size, grain density, grain sorting, grain morphology, solubility in water, and/or grain composition, and/or due to their chemistry including but not limited to their isotopic signature, adhesion, and surface energy components. These types of nutrients may originate from different sources, either from targeted mining practices and/or byproducts of needed industrial products that are all healthy, reasonably priced, and favorable to being introduced to the aquatic environment and/or for consumption.

The desired organisms may include one or more aquatic organisms. In an embodiment, the organisms may include one or more plankton types. These plankton may be of different or similar types, including but not limited to phytoplankton, cyanobacteria, zooplankton, autotrophs, diatoms, coccolithophorids, dinoflagellates, foraminifera, grazers, autotrophs, and so forth. In an embodiment, the organisms include microalgae, such as diatoms and/or coccolithophorids. The one or more plankton types may be chosen based on visible characteristics, or genetic characteristics, or other characteristics, any combination of characteristics, or chosen by algorithm, or chosen randomly, or chosen by some other process. The one or more plankton types may be chosen due to their characteristics in single-species populations and/or due to their characteristics when interacting with other organisms and/or to the water column. For example, certain types of cyanobacteria (e.g., Prochlorococcus) may be chosen due to dominance in open ocean waters and/or their ability to perform photosynthesis, particularly when grown for distribution into aquatic environments for bioremediation purposes and/or carbon removal. As another example, certain types of cyanobacteria (e.g., Spirulina) may be chosen due to nutritional value, particularly when grown for consumption by humans and/or other animals, such as cattle. As another example, various types of eukaryotic phytoplankton (e.g., diatoms, coccolithophorids, dinoflagellates) may be chosen due to tolerance for growing conditions in photobioreactors and efficiency in carbon export, particularly when grown for distribution into aquatic environments for carbon removal.

The desired organisms may include plankton and/other organisms created with biotechnology, genetic modification, gene editing, or other techniques that enable the change, creation, limitation, and/or increase in single or multiple traits, characteristics, behaviors, activities, growth rates, compositions, sinking rates, nutrient uptake, nutrient release, and/or interaction with other biologics or non-biologics. The desired organisms may include organisms intentionally bred for specific characteristics, and/or species created and/or modified using processes that can alter their characteristics, genome, or other factors, such as but not limited to bioengineering, genetic modification, or gene editing.

The desired organisms may be chosen due to biomass volume growth rates, marine biogenic calcification processes via calcifying organisms, competitive dominance within phytoplankton communities, large primary productivity contributor to oceans, light scattering versus absorption rates, defensive mechanisms from grazing activity, presence/absence of toxic species, ratio of nitrate to ammonium uptake, sunlight requirements, cell density and wall structure, cell morphology including but not limited to size, cell size, aggregate sinking speeds, bloom forming capabilities, species growth rates, efficiency of light conversion into biomass, sinking rates including but not limited to the presence of calcite and/or calcium carbonate plates and/or scales and/or silica or opal cell walls, whether remineralization process increases or decreases sinking rates, nutrient uptake and/or transport efficiency, carbon-fixing ability, carbon export ability, adaptability to changing CO₂ concentrations, ability to outcompete against other organisms, and/or how significant they are to primary productivity.

The bioreactor system may be located off-shore, including on an aquatic vessel (e.g., a traveling vessel, such as passenger ship, cruise ship, ferry, fishing boat; a fixed or anchored vessel, such as a dock that extends over a volume of water or a floating structure). Thus, the bioreactor system may be located off-shore during a growth phase (e.g., growth of the desirable organisms) and during a distribution phase (e.g., distribution into the aquatic environment). However, it should be appreciated that the photobioreactor system may be located on-shore (e.g., a warehouse on-shore) during the growth phase and then transferred to any suitable vessel (e.g., an aquatic vessel, a land-based vessel, an aircraft) that is capable of carrying and/or distributing the desired organisms during the distribution phase.

The bioreactor system may be located in saltwater settings (e.g., oceans or other saltwater environments). Salt in saltwater provides dissolved sulfate, which may be used by bacteria as a source of energy. Accordingly, deployment in the saltwater settings may further reduce cycling of methane back into the atmosphere, as compared to freshwater settings (e.g., freshwater lakes, rivers, ponds, or other freshwater environments). Further, the deployment in the saltwater settings may be preferable or provide certain advantages in carbon removal as compared to freshwater settings. Indeed, certain freshwater settings, such as rivers and streams, may produce more carbon dioxide than they remove from the atmosphere, and thus, may not be preferred deployment locations for carbon removal. For example, once organic carbon is precipitated in rivers and streams, high flow rates in rivers and streams may negatively impact carbon removal solutions, as well as result in lack of water clarity and/or a wide range of present carbon compounds. However, it should be appreciated that the bioreactor system may be located in freshwater settings for any of a variety of purposes, including carbon removal techniques, but deployment in freshwater settings may be best suited for bioremediation.

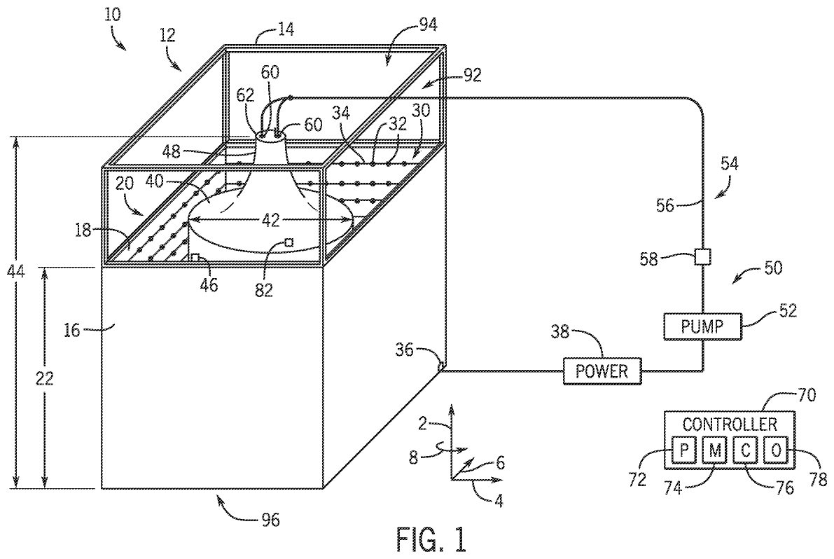

With the preceding in mind, FIG. 1 is a perspective view of an embodiment of a photobioreactor system 10 (e.g., low-cost bioreactor system). The photobioreactor system 10 and its components may be described with reference to a vertical axis or direction 2, a first lateral axis or direction 4, a second lateral axis or direction 6, and a circumferential axis or direction 8. The photobioreactor system 10 includes a frame assembly 12 (e.g., housing) that includes a frame 14. The frame 14 may include multiple vertical and horizontal bars joined together to form an open box structure (e.g., open frame structure; open sides, top, and/or bottom). The frame 14 may be formed from any suitable materials, including metal, plastic, and so forth. Further, the frame 14 may be formed by joining sections of bars together, such as via fasteners (e.g., welds, threaded bolts, threaded screws, adhesives) and/or via interference fit (e.g., key-slot interface). In certain embodiments, the frame 14 may be formed from Polyvinyl Chloride (PVC) pipe sections and fittings that are joined together via interference fit, which may be supplemented or further secured via adhesive. For example, multiple vertical and horizontal bars of PVC pipe sections and PVC elbow fittings may be joined together via interference fit, as well as via adhesive.

The frame assembly 12 also includes solid wall sections 16. In some embodiments, the solid wall sections 16 may be formed from at least one flexible sheet (e.g., paper, film) with at least one reflective surface 18 (e.g., foil surface or reflective insulation) that faces toward an interior chamber 20 defined by the frame assembly 12. In such cases, the solid wall sections 16 may be coupled to the frame 14 via wrapping the at least one flexible sheet about the vertical bars of the frame 14 or otherwise coupling the at least one flexible sheet to the frame 14 such that the at least one reflective surface 18 faces toward the interior chamber 20. It should be appreciated that the solid wall sections 16 may be formed by a single flexible sheet that extends circumferentially about the interior chamber 20 or separate sections formed by separate flexible sheets arranged to extend circumferentially about the interior chamber 20. In any case, the solid wall sections 16 (e.g., four solid wall sections 16) extend circumferentially about the interior chamber 20 to form a reflective barrier or wall about the interior chamber 20. It should be appreciated that the at least one flexible sheet may wrap around and/or couple to outer edges of the vertical bars of the frame 14 and may leave certain portions of the vertical bars exposed to the interior chamber 20. Accordingly, reflective tape (e.g., foil tape) or other reflective material may be applied to the portions of the vertical bars that are exposed to the interior chamber 20 to thereby form the reflective barrier or wall about the interior chamber 20.

The frame assembly 12 also includes a light system 30 with one or more light sources 32. For example, the light system 30 may include the one or more light sources 32 arranged along one or more cables 34 (e.g., strings). Thus, the light system 30 may be efficiently applied to the at least one reflective surface 18 of the solid wall sections 16 by wrapping the one or more cables 34 circumferentially about the interior chamber 20 and then coupling the one or more cables 34 to the solid wall sections 16 via fasteners (e.g., welds, threaded bolts, threaded screws, adhesives). In some embodiments, the one or more cables 34 may be a single cable or multiple cables arranged in series to form a continuous cable assembly about the interior chamber 20. As shown, the light system 30 may be arranged to place the one or more light sources 32 in discrete rows that are spaced apart (e.g., extend horizontally; spaced apart vertically) between a bottom portion or edge of the interior chamber 20 and an upper portion or edge of the interior chamber 20. Further, the light system 30 may include any suitable number of light sources 32 with any suitable characteristics (e.g., emit white light; full spectrum; mimic sunlight). The light system 30 may include a connector 36 that is configured to connect to a power source 38.

As shown, the interior chamber 20 is configured to receive and/or support a container 40 (e.g., tank). The container 40 may be formed from any suitable material, such as plastic, glass, and so forth. The container 40 may be formed from a transparent material that allows light (e.g., from the one or more light sources 32 and/or ambient light, including sunlight) to pass through the container 40. In some embodiments, the transparent material may provide permit 80 to 93 percent, 85 to 90 percent, or about 90 percent of visible light to pass through a wall or external shell of the container. In some embodiments, the transparent material may provide light transmittance values of 1200 to 1500 lux (e.g., measured within the container 40 at a distance of 5 to 45 centimeters (cm) from the wall or external shell of the container 40; without the contents in the container 40). Further, a dimension 42 (e.g., diameter; width; along the first lateral axis 4 and along the second lateral axis 6) of the container 40 may be equal to or less than approximately 50 centimeters (cm), 40 cm, 30 cm, or 35 cm. In some embodiments, the dimension 42 of the container 40 may be between approximately 12 to 50 centimeters (cm), 20 to 40 cm, 25 to 35 cm, or 27 to 30 cm. The dimension 42 may be selected to enable light penetration to an interior (e.g., center) of the container 40 to promote growth of desired organisms in the container 40, such as even when the container 40 is filled with contents (e.g., the desired organisms, nutrients, water).

A height 44 of the container 40 may be equal to or less than approximately 5 meters (m), 4 m, 3 m, 2 m, 1.5 m, 1.3 m, or 1 m. In some embodiments, the height 44 of the container 40 may be between approximately 1 to 5 meters (m), 1 to 4 m, 1 to 3 m, 1 to 2 m, or 1.3 to 1.5 m. The height 44 may be selected to facilitate lifting and/or transferring the container 40, such as even when the container 40 is filled with the contents. In some embodiments, the container 40 may include a handle 46 and/or a neck portion 48 to facilitate lifting and/or transferring the container 40. However, it should be appreciated that the container 40 may have any of a variety of shapes, sizes, and/or features (e.g., a cylindrical base with the neck portion 48, as shown in FIG. 1; entirely cylindrical without the neck portion 48; with or without the handle 46). Further, a height 22 of the solid wall sections 16 may be selected to enable the solid wall sections 16 to circumferentially surround at least a portion of the container 40 (e.g., a cylindrical base portion of the container 40;

an entirety of the container 40; at least 75, 80, 85, 90, or 95 percent of the height 44 of the container 40).

The container 40 may have a capacity equal to or less than approximately 0.2 cubic meters (m³), 0.1 m³, 0.05 m³, 0.04 m³, 0.03 m³, 0.025 m³, 0.02 m³, 0.015 m³, or 0.01 m³. In some embodiments, the container 40 may have a capacity of between approximately 0.01 to 0.2 cubic meters (m³), 0.01 to 0.1 m³, or 0.015 to 0.05 m³. As described herein, the container 40 may receive and hold the contents to promote growth of the desired organisms, which may include one or more plankton types. The contents may also include nutrients, such as silica, iron, cobalt, copper, aluminum, nitrogen, phosphorous, magnesium, manganese, calcium, sodium, potassium, and carbon, all in various forms and solutions. In some embodiments, the nutrients may include volcanic ash or synthetic volcanic ash. The nutrients may be added to the containers 40 as dry compositions (e.g., powders) and/or the nutrients may be added to the containers 40 as part of fluid mixtures (e.g., with environmental water). For example, the environmental water may be selected to match (e.g., within 10 percent) a density of a destination (e.g., for deposition of the contents of the container 40) and/or may be retrieved from the destination.

The photobioreactor system 10 may also include an air system 50. The air system 50 includes a pump 52 (e.g., air pump) and a tubing assembly 54. The tubing assembly 54 includes tubing 56 and a backflow preventer 58, wherein the tubing 56 extends from the pump 52 and into the container 40. For example, the tubing 56 may pass through one or more openings 60 formed in a lid 62 (e.g., cover) of the container 40. As shown, the tubing 56 may bifurcate into multiple tubing sections (e.g., two tubing sections) that pass through respective openings 60 formed in the lid 62 of the container 40. However, it should be appreciated that the air system 50 may have any of a variety of parts, configurations, and/or features (e.g., separate pieces of tubing 56 that each extend from the pump 52 through respective openings 60 formed in the lid 62 of the container 40). In certain embodiments, the lid 62 of the container 40 is not airtight, which may facilitate additional air flow into the container 40 (e.g., to support growth of the desired organism in the container 40).

The photobioreactor system 10 may also include a controller 70 (e.g., electronic controller) that includes a processor 72, a memory device 74, a communication device 76, and/or an output device 78. The controller 70 may control certain operations of the photobioreactor system 10. The processor 72 may be any suitable type of computer processor or microprocessor capable of executing computer-executable code. In certain embodiments, the processor 72 may also include multiple processors that may perform the operations described herein, and certain operations may be distributed between the processor 72 and one or more remote servers (e.g., controller 70 may be a distributed controller with components located both locally at the container 40 and remotely from the container 40 to distribute processing to carry out techniques disclosed herein). The memory device 74 may be any suitable articles of manufacture that can serve as media to store processor-executable code, data, or the like. These articles of manufacture may represent computer-readable media (e.g., any suitable form of memory or storage) that may store the processor-executable code used by the processor 72 to perform the presently disclosed techniques. The memory device 74 may also be used to store data, various other software applications, and the like. The memory device 74 may represent non-transitory computer-readable media (e.g., any suitable form of memory or storage) that may store the processor-executable code used by the processor 72 to perform various techniques described herein. The communication device 75 may facilitate communication with other devices and/or systems. The output device 78 may include a display screen, a speaker, and/or any other suitable device that is configured to provide information, alerts, and/or notifications. It should be appreciated that the output device 78 may also be configured to operate as an input device (e.g., touch screen display screen), or the controller 70 may include or be coupled to a separate input device.

The controller 70 may be configured to control operational parameters of the light system 30 and/or the air system 50. The controller 70 may be configured to provide power to illuminate the one or more light sources 32 according to a schedule (e.g., on/off schedule, such as on for 20 hours and off for 4 hours on a repeating schedule). It is presently recognized that continuous light may result in fastest growth in the container 40, but may negatively affect transfer and behavior in the aquatic environment for the environmental treatment processes. Accordingly, the schedule may include at least some time without light from the one or more light sources 32 to mimic night/darkness of the aquatic environment and to support the transfer to the aquatic environment. However, the desired organisms that are targeted for consumption may be exposed to continuous light or at least a different schedule.

The controller 70 may be configured to receive inputs that indicate and/or enable the controller 70 to determine the operational parameters (e.g., adjustments and/or appropriate target operational parameters). For example, the operator may provide the inputs of the operational parameters. As another example, the operator may provide the inputs indicative of a type of the desired organisms in the container 40. Then, the controller 70 may access a database that stores (e.g., in a lookup table) the appropriate target operational parameters for the type of the desired organisms in the container 40, and the controller 70 may control the light system 30 and/or the air system 50 accordingly. As noted herein, the desired organisms may include one or more aquatic organisms. In an embodiment, the organisms may include one or more plankton types. These plankton may be of different or similar types, including but not limited to phytoplankton, cyanobacteria, zooplankton, autotrophs, diatoms, coccolithophorids, dinoflagellates, foraminifera, grazers, autotrophs, and so forth.

The controller 70 may be configured to receive sensor inputs indicative of the growth of the desired organisms in the container 40 and/or other inputs indicative of the growth of the desired organisms in the container 40. For example, the photobioreactor system 10 may include a sensor 82, such as a fluorometer that measures phycocyanin, phycoerythrin, and/or chlorophyll, positioned at the container 40. The sensor 82 may provide the sensor inputs indicative of the growth of the desired organisms in the container 40, and the controller 70 may be configured to process the sensor inputs to determine the operational parameters. For example, in response to slow and/or no growth, the controller 70 may adjust the light system 30 to provide additional light over time (e.g., from 20 hours to 21 hours) and/or adjust the air system 50 to increase a speed of the pump 52 to increase oxygen and/or agitation provided via the air system 50. The photobioreactor system 10 may include any of a variety of sensors, including sensors that monitor pH, dissolved oxygen, temperature, and/or pressure within the container 404. As

In some embodiments, the controller 70 may provide recommendations and/or alerts (e.g., text messages on the output device 78) based on the inputs from the operator, the sensor inputs, and/or other information (e.g., stored programs and schedules). The controller 70 may provide the recommendations and/or the alerts to notify the operator of the appropriate target operational parameters (e.g., recommended nutrients to add to the container 40). The controller 70 may provide the recommendations and/or the alerts to indicate organism growth information (e.g., appropriate growth, fast growth, slow growth, no growth), nutrient information (e.g., appropriate type(s) and/or amount(s) of nutrient(s) to add to the container 40), water information (e.g., appropriate type(s) and/or amount(s) of water to add to the container 40; to achieve a target density that matches the environmental water), and so forth. It should be appreciated that the controller 70 may provide the recommendations and/or the alerts via the output device 78, which may be located locally at (e.g., proximate to) the container 40 and/or located remotely from the container 40 (e.g., on-shore laboratory). Indeed, any information, inputs, and/or outputs may be provided from, provided to, and/or exchanged between components located locally at the container 40 and other components located remotely from the container 40 to enable the operator to monitor conditions locally at the container 40 without being present locally at the container 40 (e.g., located remotely from the container 40; the container 40 is off-shore or near-shore, such as on a vessel or in a warehouse near a port or a dock, and the output device 78 is part of a computing device in an on-shore in a laboratory and/or is part of a portable computing device carried by the operator). It should be appreciated that the sensor 82 may also include its own communication device that communicates the sensor data directly (e.g., wirelessly) to a computing device that is located remotely from the container 40. Thus, the operator may be aware of the recommendations and/or the alerts to enable the operator to take certain actions to support growth of the desired organisms in the container 40.

In some embodiments, the controller 70 may be communicatively coupled to valves, pumps, and so forth to enable the controller 70 to provide control signals to provide additional desired organisms, nutrients, and/or water to the container 40 based on analysis of the inputs from the operator, the sensor inputs, and/or the other information. Thus, the photobioreactor system 10 may have any suitable level of automation and/or operator involvement to facilitate growth of the desired organisms. Advantageously, a structure of the photobioreactor system 10 and/or control features provided by the controller 70 enable the photobioreactor system 10 to grow a wide variety of desired organisms in a wide variety of nutrients for a wide variety of purposes. Indeed, the photobioreactor system 10 may grow certain types of desired organisms with certain nutrient mixes for treatment of bodies of water, and the photobioreactor system may also grow other types of desired organisms with other nutrient mixes for consumption. Further, the nutrient mixes, the operational parameters of the light system 130, and/or the operational parameters of the air system 150 may be selected based on the desired organisms (e.g., to provide optimal growth rates in the containers 40 and/or to support favorable results and end uses).

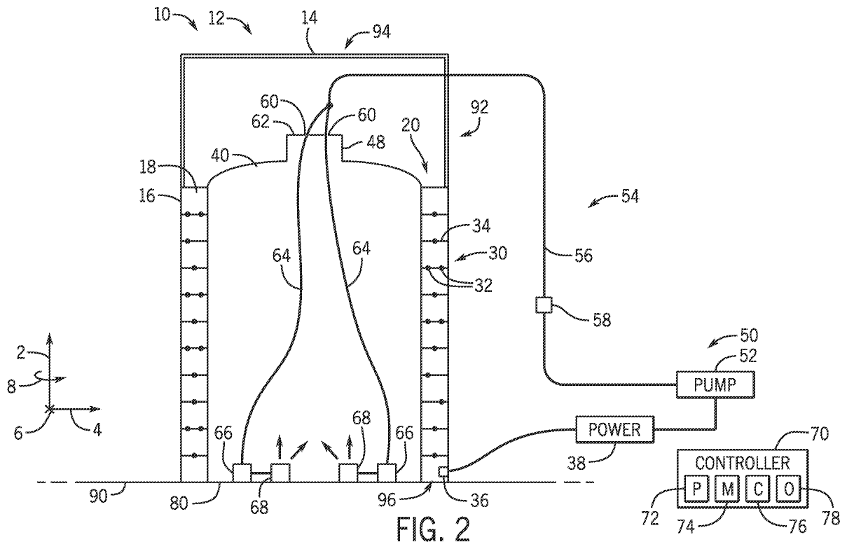

FIG. 2 is a cross-sectional side view of an embodiment of the photobioreactor system 10. As shown, the photobioreactor system 10 includes the frame assembly 12 with the frame 14, the solid wall sections 16 formed with the at least one reflective surface 18 facing toward the interior chamber 20, and the light system 30 with the one or more light sources 32 on the one or more cables 34. Additionally, the photobioreactor system 10 includes the air system 50 with the pump 52 and the tubing assembly 54 with the tubing 56 and the backflow preventer 58. In FIG. 2, the tubing 56 splits into the multiple tubing sections 64 that pass through the respective openings 60 formed in the lid 62 of the container 40. Each of the multiple tubing sections 64 is coupled to a weight 66 and an air distribution device 68 (e.g., one or more air stones). The weight 66 may be formed from any suitable material, such as metal, that is heavy enough to draw the respective tubing section 64 and the respective air distribution device 68 toward a bottom surface 80 of the container 40. In some embodiments, the air system 50 may be configured (e.g., via lengths of the multiple tubing sections 64 and the weights 66) to position the air distribution device 68 on the bottom surface 80 of the container 40. The air distribution device 68 may be formed from any suitable material, such as a porous stone, bonded glass beads, synthetics (e.g., fiberglass), and so forth, to enable distribution of the air from the pump 52 into the contents in the container 40. In some embodiments, the air system 50 provides mixing and agitation to the contents in the container 40 (e.g., via the distribution of the air from the air distribution devices 68), and the photobioreactor system 10 does not include any agitators other than the air distribution devices 68 at distal ends of the multiple tubing sections 64.

Additional features of the photobioreactor 10 may be understood with reference to FIGS. 1 and 2. For example, as noted herein, the frame 14 provides the open box structure that supports the solid wall sections 16. The open box structure may be open at sides of the frame 14, a top of the frame 14, and/or a bottom of the frame 14. For example, the open box structure may enable an operator (e.g., a human operator) to view at least a portion of the container 40 (e.g., upper portion or surface and/or the neck portion 48) while the container 40 is positioned in the interior chamber 20. Accordingly, the operator may be able to visually inspect and/or assess the contents of the container 40 without manipulating the container 40 or the frame assembly 12. Further, the operator may be able to perform maintenance operations, such as adjusting (e.g., adding and/or removing) the contents in the container 40 and/or adjusting the air system 50 (e.g., removing components from the interior of the container 40 for inspection, replacement, and/or repair), without manipulating the frame assembly 12.

In some embodiments, the open box structure may enable the operator to lift the container 40 from the interior chamber 20 by hoisting or raising the container 40 vertically relative to the frame assembly 12 and through the top of the open box structure. Accordingly, the operator may be able to retrieve or remove the container 40 from the frame assembly 12 to facilitate various operations, including to visually inspect and/or assess the contents of the container 40, perform the maintenance operations, and/or transfer the container 40 (e.g., to an aquatic environment for distribution of the contents of the container 40). In some embodiments, the open box structure may enable the operator to lift the frame assembly 12 by hoisting or raising the frame assembly 12 vertically relative to the container 40 due the open box structure along the bottom of the frame 14 (e.g., the container 40 rests directly on a ground surface 90). Accordingly, the operator may be able to retrieve or remove the frame assembly 12 away from the container 40 to facilitate various operations, including to visually inspect and/or assess the contents of the container 40, perform the maintenance operations (including on the frame assembly 12, such as to replace the one or more light sources 32), and/or to facilitate transfer of container 40 (e.g., lifting from the ground surface 90 via a forklift). In particular, with reference to FIGS. 1 and 2, the open box structure may provide various access points including open side windows 92 (e.g., openings) that are vertical gaps between upper-most horizontal bars of the frame 14 and upper-most edges of the solid wall sections 16, an open top window 94 (e.g., opening), and/or an open bottom window 96 (e.g., opening).

In some embodiments, the frame assembly 12 with the container 40 therein may be considered to form a photobioreactor module (e.g., block). In such cases, the photobioreactor module may be stacked vertically with one or more additional bioreactor modules. For example, with reference to FIGS. 1 and 2, a support plate may be placed over (e.g., to cover) the open top window 94 of the frame assembly 12, then an additional frame assembly may be positioned on the support plate, and then an additional container may be placed within the additional frame assembly, and so forth. Advantageously, in such cases, the multiple bioreactor modules may enable visual inspection and/or access to each of the containers via respective open side windows provided at each of the multiple frame assemblies. It should be appreciated that the multiple bioreactor modules may be separate modules or may be formed as one larger systems (e.g., the light system is shared between multiple vertical levels of the multiple bioreactor modules), the frame assembly 12 may include a different structure and/or fasteners (e.g., welds), and so forth depending on preferred constructions parameters (e.g., stability versus adjustability).

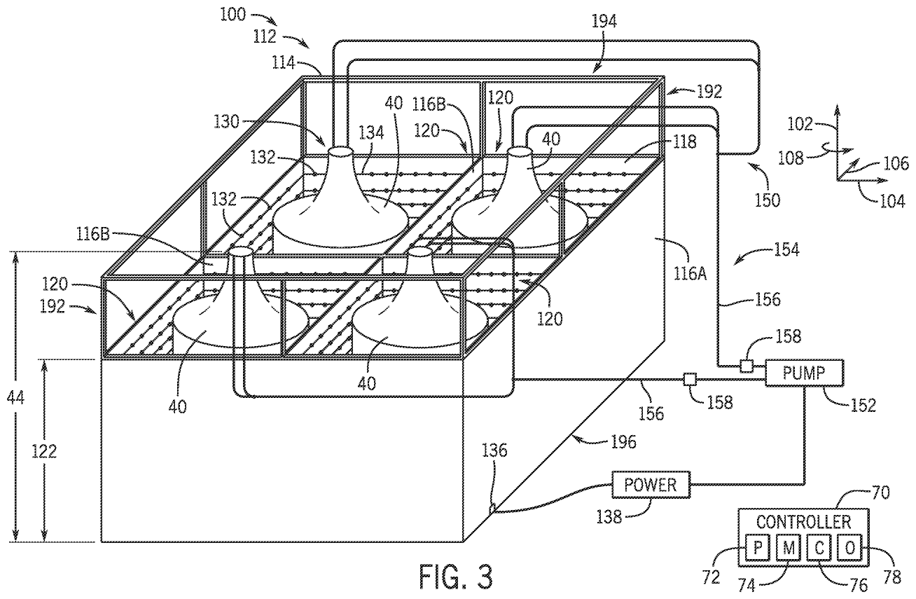

FIG. 3 is a perspective view of an embodiment of a photobioreactor system 100 (e.g., a low-cost bioreactor system). The photobioreactor system 100 and its components may be described with reference to a vertical axis or direction 102, a first lateral axis or direction 104, a second lateral axis or direction 106, and a circumferential axis or direction 108. The photobioreactor system 100 includes a frame assembly 112 that includes a frame 114. The frame 114 may include multiple vertical and horizontal bars joined together to form an open box structure (e.g., open frame structure; open sides, top, and/or bottom). The frame 114 may be formed from any suitable materials, including metal, plastic, and so forth. Further, the frame 114 may be formed by joining sections of bars together, such as via fasteners (e.g., welds, threaded bolts, threaded screws, adhesives) and/or via interference fit (e.g., key-slot interface). In certain embodiments, the frame 114 may be formed from PVC pipe sections and fittings that are joined together via interference fit, which may be supplemented or further secured via adhesive. For example, multiple vertical and horizontal bars of PVC pipe sections and PVC elbow fittings may be joined together via interference fit, as well as via adhesive.

The frame assembly 112 also includes solid wall sections 116. In some embodiments, the solid wall sections 116 may be formed from at least one flexible sheet (e.g., paper, film) with at least one reflective surface 118 (e.g., foil surface) that faces toward interior chambers 120 defined by the frame assembly 112. For example, in FIG. 3, four interior chambers 120 are defined by the frame assembly 112. However, it should be appreciated that any number of interior chambers 120 may be defined by the frame assembly 112 (e.g., 2, 3, 4, 5, 6, 7, 8, 9, 10, 50, 100, or more). Further, while the frame assembly 112 in FIG. 3 is a square open box structure with four interior chambers 120 arranged in a 2×2 grid, it should be appreciated that the frame assembly 112 may have any shape open box structure (e.g., rectangular) with any number of interior chambers 120 in any arrangement (e.g., 2×1, 3×1, 3×2, 3×3, 4×1, 4×2, 4×3, 4×4, 5×1, 5×2, 5×3, 5×4, 5×5, and so forth). Generally, it may be desirable for the frame assembly 112 to enable access to all of the interior chambers 120, and thus, the frame assembly 112 may arrange the interior chambers 120 in grids of one or two rows (e.g., N×1 or N×2, where N is any integer) so that the operator may visually inspect and/or readily access each of the interior chambers 120 while the operator is positioned at the frame assembly 112 (e.g., standing adjacent to and/or walking about the frame assembly 112).

In FIG. 3, the solid wall sections 116 may include exterior solid wall sections 116A and interior solid wall sections 116B. The exterior solid wall sections 116A may be coupled to the frame 114 via wrapping the at least one flexible sheet about the vertical bars of the frame 114 or otherwise coupling the at least one flexible sheet to the frame 114 such that the at least one reflective surface 118 faces toward the interior chambers 120. For example, the exterior solid wall sections 116A may be formed by a single flexible sheet that extends circumferentially about all of the interior chambers 120 or separate sections formed by separate flexible sheets arranged to extend circumferentially about all of the interior chambers 120. In any case, the exterior solid wall sections 116A extend circumferentially about all of the interior chambers 20. It should be appreciated that the at least one flexible sheet may wrap around and/or couple to outer edges of the vertical bars of the frame 114 and may leave certain portions of the vertical bars exposed to the interior chambers 120. Accordingly, reflective tape or other reflective material may be applied to the portions of the vertical bars that are exposed to the interior chambers 120 to thereby form a continuous reflective barrier or wall about all the interior chambers 20.

The interior solid wall sections 116B may be formed in any of a variety of manners. For example, the interior solid wall sections 116B may include four flexible sheets that each include two reflective surfaces (e.g., opposite sides), and the four flexible sheets are mounted to a rigid base (e.g., boards) and arranged in a cross shape (or an interior grid shape, for more than four interior chambers) that is sized to fit within the exterior solid wall sections 116A to form the interior chambers 120. In some embodiments, the interior solid wall sections 116B are separate and removable from the exterior solid wall sections 116A, such as to facilitate maintenance operations. For example, the operator may lift out the interior solid wall sections 116B as a unit without disturbing or interfering with the exterior solid wall sections 116A. However, the interior solid wall sections 116B may be coupled to the exterior solid wall sections 116A, such as via fasteners (e.g., adhesive), or may be integrally formed with the exterior solid wall sections 116A (e.g., one-piece). In any case, the interior chambers 120 are circumferentially surrounded by the exterior solid wall sections 116A and the interior solid wall sections 116B, and thus, each of the interior chambers 120 is circumferentially surrounded by the at least one reflective surface 118.

The frame assembly 112 also includes a light system 130 with one or more light sources 132. For example, the light system 130 may include the one or more light sources 132 arranged along one or more cables 134 (e.g., strings). Thus, the light system 130 may be efficiently applied to the at least one reflective surface 118 of the solid wall sections 116 by wrapping the one or more cables 134 circumferentially about the interior chambers 120 and then coupling the one or more cables 134 to the solid wall sections 116 via fasteners (e.g., welds, threaded bolts, threaded screws, adhesives). In some embodiments, the one or more cables 134 may be a single cable or multiple cables arranged in series to form a continuous cable assembly about the interior chambers 120. However, a variety of other arrangements are envisioned. For example, the one or more cables 134 may include a single cable or series of cables arranged about the exterior solid wall sections 116A, and then a separate single cable or series of cables arranged about the interior solid wall sections 116B. As shown, the light system 130 may be arranged to place the one or more light sources 132 in discrete rows that are spaced apart (e.g., extend horizontally; spaced apart vertically) between a respective bottom portion or edge of each of the interior chambers 20 and a respective upper portion or edge of each of the interior chambers 20. Further, the light system 130 may include any suitable number of light sources 132 with any suitable characteristics (e.g., emit white light; full spectrum; mimic sunlight). The light system 130 may include at least one connector 136 that is configured to connect to a power source 138.

As shown, the interior chambers 20 are configured to receive and/or support multiple containers 40. The containers 40 may have any properties described with reference to FIGS. 1 and 2. As shown, a height 122 of the solid wall sections 116 may be selected to enable the solid wall sections 116 to circumferentially surround at least a portion of the container 40 (e.g., a cylindrical base portion of the container 40; an entirety of the container 40; at least 75, 80, 85, 90, or 95 percent of the height 44 of the container 40).

The photobioreactor system 110 may also include an air system 150. The air system 150 includes a pump 152 (e.g., air pump) and a tubing assembly 154. The tubing assembly 154 includes tubing 156 and at least one backflow preventer 158, wherein the tubing 156 extends from the pump 152 and into the containers 40. For example, the tubing 156 may pass through the one or more openings formed in the lids of the containers 40, as described with reference to FIGS. 1 and 2. As shown, the tubing 156 include two pieces of tubing 156 that extend from the pump 152 and that each bifurcate (e.g., one or more times) to provide multiple tubing sections (e.g., two tubing sections) that pass through respective openings formed in the lids of the containers 40. However, it should be appreciated that the air system 150 may have any of a variety of parts, configurations, and/or features. Further, each of the multiple tubing sections within the containers 40 may include a respective weight and a respective air distribution device, as described with reference to FIG. 2. The photobioreactor system 100 may also include the controller 70 (e.g., electronic controller) that includes the processor 72, the memory device 74, the communication device 76, and/or the output device 78.

As noted herein, the frame 114 provides the open box structure that supports the solid wall sections 116. The open box structure may be open at sides of the frame 114, a top of the frame 114, and/or a bottom of the frame 114. Thus, the open box structure may enable the operator to view at least respective portions of the containers 40 (e.g., upper portions or surfaces and/or the neck portions) while the containers 40 are positioned in the interior chambers 120. Accordingly, the operator may be able to visually inspect and/or assess the contents of the containers 40 without manipulating the containers 40 or the frame assembly 112. Further, the operator may be able to perform maintenance operations, such as adjusting (e.g., adding and/or removing) the contents in the containers 40 and/or adjusting the air system 150 (e.g., removing components from the interior of the containers 40 for inspection, replacement, and/or repair), without manipulating the frame assembly 112.

In some embodiments, the open box structure may enable the operator to lift the containers 40 from the interior chambers 20 by hoisting or raising the containers 40 vertically relative to the frame assembly 112 and through the top of the open box structure. Accordingly, the operator may be able to retrieve or remove the containers 40 from the frame assembly 112 to facilitate various operations, including to visually inspect and/or assess the contents of the containers 40, perform the maintenance operations, and/or transfer the containers 40 (e.g., to an aquatic environment for distribution of the contents of the containers 40). In some embodiments, the open box structure may enable the operator to lift the frame assembly 112 (or separate portions thereof, such as the exterior solid sections 116A and/or the interior solid wall sections 116B) by hoisting or raising the frame assembly 112 (or the separate portions thereof) vertically relative to the containers 40. Accordingly, the operator may be able to retrieve or remove the frame assembly 112 (or the separate portions thereof) away from the containers 40 to facilitate various operations, including to visually inspect and/or assess the contents of the containers 40, perform the maintenance operations (including on the frame assembly 112, such as to replace the one or more light sources 132), and/or to facilitate transfer of container 140 (e.g., lifting from the ground surface via a forklift). In particular, with reference to FIG. 3, the open box structure may provide various access points including open side windows 192 that are vertical gaps between upper-most horizontal bars of the frame 14 and upper-most edges of the exterior solid wall sections 116A, an open top window 194, and/or an open bottom window 196.

In some embodiments, the frame assembly 112 with the containers 40 therein may be considered to form have multiple bioreactor modules (e.g., blocks). In such cases, the photobioreactor modules may be stacked vertically with one or more additional bioreactor modules. For example, with reference to FIG. 3, a support plate may be placed over (e.g., to cover) the open top window 194 of the frame assembly 112, then an additional frame assembly may be positioned on the support plate, and then additional containers may be placed within the additional frame assembly, and so forth. Advantageously, in such cases, the multiple bioreactor modules may enable visual inspection and/or access to each of the containers via respective open side windows provided at each of the multiple frame assemblies. It should be appreciated that the multiple bioreactor modules may be separate modules or may be formed as one larger systems (e.g., the light system is shared between multiple vertical levels of the multiple bioreactor modules), the frame assembly 112 may include a different structure and/or fasteners (e.g., welds), and so forth depending on preferred constructions parameters (e.g., stability versus adjustability).

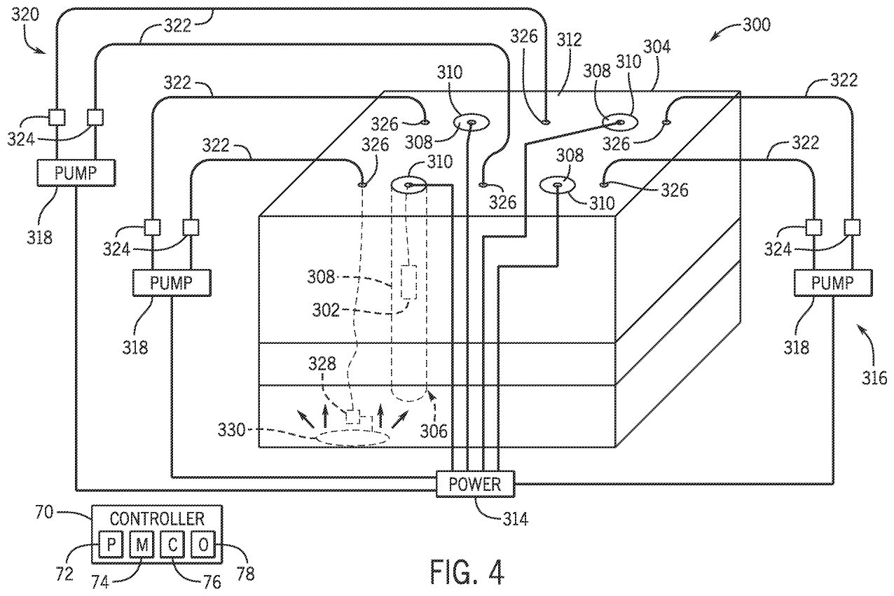

FIG. 4 is a perspective view of an embodiment of a photobioreactor system 300 with at least one light source 302 positioned within a container 304, in accordance with embodiments the present disclosure. The container 304 may have a capacity equal to or greater than approximately 4 cubic meters (m³), 3.5 m³, 3 m³, 2.5 m³, 2 m³, 1.5 m³, 1 m³, 0.8 m³, or 0.3 m³. In some embodiments, the container 304 may have a capacity of between approximately 0.3 to 4 cubic meters (m³), 0.8 to 3.5 m³, 1 to 3 m³, 1.5 to 2 m³. Accordingly, the container 304 may be larger than the container 40 shown in FIGS. 1-3 (e.g., 25 to 100 times larger).

The photobioreactor system 300 may include a light system 306, which has at least one transparent light tube 308 that houses at least one respective light source 302. As shown, the light system 306 includes four transparent light tubes 308 that each house at least one respective light source 302; however, the photobioreactor system 300 may include any suitable number of transparent light tubes 308 and/or light sources 302 (e.g., 1, 2, 3, 4, 5, 6, or more) depending on a size of the container 304 and/or other parameters (e.g., appropriate light conditions for desired organisms in the container 304). It should be appreciated that a number of transparent light tubes 308 and/or the light sources 302 may vary and/or be selected based on dimensions (e.g., a length and a width) of the container 304, as well as based on a density of the light sources 302 and/or characteristics (e.g., intensity) of light output by the light sources 302. For example, the number of transparent light tubes 308 may be selected such that the transparent light tubes 308 have a spacing equal to or less than approximately 50 centimeters (cm), 45 cm, 40 cm, 35 cm, 30 cm, 25 cm, or 20 cm, or with the spacing between approximately 20 to 50 cm or 30 to 40 cm. In some embodiments, each transparent light tube 208 may include multiple light sources 302, and the multiple light sources 302 may be distributed about an interior surface of the transparent light tube 208 (e.g., wrapped circumferentially about the interior surface and spaced apart, such as by a few centimeters, between a top end portion and a bottom end portion of the transparent light tube 208). Further, the one or more transparent light tubes 308 may be at any position and/or arranged in any configuration (e.g., form corners of a square configuration). Further, each of the at least one transparent light tubes 308 may be supported within and/or suspended from a respective opening 310 in a lid 312 of the container 304. Each light source 302 is coupled to a power source 314.

Additionally, the photobioreactor system 300 may include an air system 316, which has at least one pump 318 and a tubing assembly 320. The tubing assembly 320 includes tubing 322 and at least one backflow preventer 324 (e.g., one per tubing 322), wherein the tubing 322 extends from the at least one pump 318 and into the container 304. For example, the tubing 322 may pass through the one or more openings 326 formed in the lid 312 of the container 304. It should be appreciated that the air system 150 may have any of a variety of parts, configurations, and/or features (e.g., bifurcations). Further, each tubing 322 within the container 304 may include a respective weight 328 and a respective air distribution device 330 (e.g., one or more air stones). As shown, the respective air distribution device 330 may include a ring of air flow devices (e.g., multiple air stones) that provide air as well as agitate (e.g., mix) the contents within the container 304. It should be appreciated that a number of air distribution devices 330 may vary and/or be selected based on dimensions (e.g., the length and the width) of the container 304, as well as based on a number of air flow devices (e.g., air stones) included in each of the air distribution devices 330. For example, with a first number of air flow devices (e.g., 1 to 4 air stones) the number of air distribution devices 330 may be selected such that the air distribution devices 330 have a spacing (e.g., between respective outer edges) equal to or less than approximately 50 centimeters (cm), 45 cm, 40 cm, 35 cm, 30 cm, 25 cm, or 20 cm, or with the spacing between approximately 20 to 50 cm or 30 to 40 cm. With a second number of air flow devices (e.g., 4 to 10 air stones), the number of air distribution devices 330 may be selected such that the air distribution devices 330 have a spacing (e.g., between respective outer edges) equal to or less than approximately 100 centimeters (cm), 90 cm, 80 cm, 70 cm, 60 cm, or 50 cm, or with the spacing between approximately 50 to 100 cm or 60 to 80 cm. Advantageously, with the one or more light sources 302 within the container 304, the container 304 may be formed from an opaque material and/or light characteristics (e.g., intensity) may be more readily adjusted to facilitate growth of the desired organisms within the container 304. The container 304 may be a photobioreactor module (e.g., block) that can be stacked and/or provided as part of a set of photobioreactor modules. The photobioreactor system 300 may include the controller 70, which is configured to control and/or determine operational parameters, as described herein.

The photobioreactor systems (e.g., the photobioreactor systems 10, 100, 300) described herein may be placed in any of a variety of settings or environments, including indoor locations, outdoor locations, on-shore locations, and/or off-shore locations. For example, the photobioreactor systems may be located in a warehouse or other building at an on-shore location. In some such cases, the containers (e.g., the containers 40, 304) of the photobioreactor systems (or the contents of the containers) may be transported from the on-shore location to a destination (e.g., a body of water for the contents to be deposited into the body of water; a processing plant to generate food for consumption, such as to farm animals or to humans).

As another example, the photo bioreactor systems may be located on a vessel at an off-shore location at a body of water. In some such cases, the contents of the containers of the photobioreactor systems may deposited into the body of water directly from the vessel. In some embodiments, the containers of the photobioreactor systems may be loaded onto a separate vessel to be transported from the vessel at the off-shore location to a destination (e.g., another connected body of water). Further, the containers of the photobioreactor systems may be part of the vessel (e.g., a marine holding tank of a boat is the container; a ballast tank of a ship is the container; coupled to supportive components, such as the light system, the air system, and/or the controller).

Advantageously, the photobioreactor systems 10, 100 facilitate efficient transport of the containers 40 (e.g., disassembly of the photobioreactor systems at an initial location and then reassembly of the photobioreactor systems on a transport vessel, such as a truck or the separate vessel; transport of the containers 40 with the air system 50, 150 intact to provide air and to agitate the contents of the containers 40 during the transport). Similarly, the photobioreactor system 300 may also facilitate efficient transport of the containers 304 (e.g., lifting as a single unit with a forklift onto a transport vessel).

As noted herein, the photobioreactor systems 10, 100, 300 may be at an on-shore location (e.g., in a warehouse) to facilitate growth of the desired organisms in the containers 40, 304. Then, the containers 40, 304 (or at least the contents of the containers 40, 304) may be transported to an aquatic environment. To disperse the contents of the containers 40, 304 into the aquatic environment, the photobioreactor systems 10, 100, 300 may include or work in conjunction with a mixing system and method. For example, the contents of the containers 40, 304 may be mixed with local water from the aquatic environment (e.g., ocean water) to form a mixed solution (e.g., the contents of the containers 40, 304 and the local water) that has a density that substantially corresponds to (e.g., equal to or below; within approximately 10, 5, 3, 2, or 1 percent) a density of the local water from the aquatic environment. In particular, a first volume (e.g., a higher volume) of the local water from the aquatic environment may be pumped into a mixing device of the mixing system, and a second volume (e.g., a lower volume) of the contents of the containers 40, 304 is also introduced into the mixing device of the mixing system. For example, the first volume and the second volume may have a ratio of approximately 25:1, 30:1, 35:1, 40:1, 45:1, 50:1, 55:1, 60:1, or any other suitable ratio that achieves a target density of the mixed solution. The mixing system may include one mixing device to mix the contents of the containers 40, 304 with the local water, or the mixing system may include one mixing device to mix the contents of the containers 40, 304 with the local water and another mixing device to separately mix additional nutrients with the local water for dispersal into the aquatic environment. In some embodiments, the mixing system may include one or more outlets to disperse the mixed solution(s) into the aquatic environment proximate to or at a water surface of a body of water of the aquatic environment, as described herein. It should be appreciated that the mixing system may be located at any suitable off-shore location, such as on an aquatic vessel. For example, the containers 40, 304 may be loaded onto the aquatic vessel, which may transport the containers 40, 304 to a target location for dispersal. Then, the mixing system and method may be carried out on the aquatic vessel at the target location for dispersal.

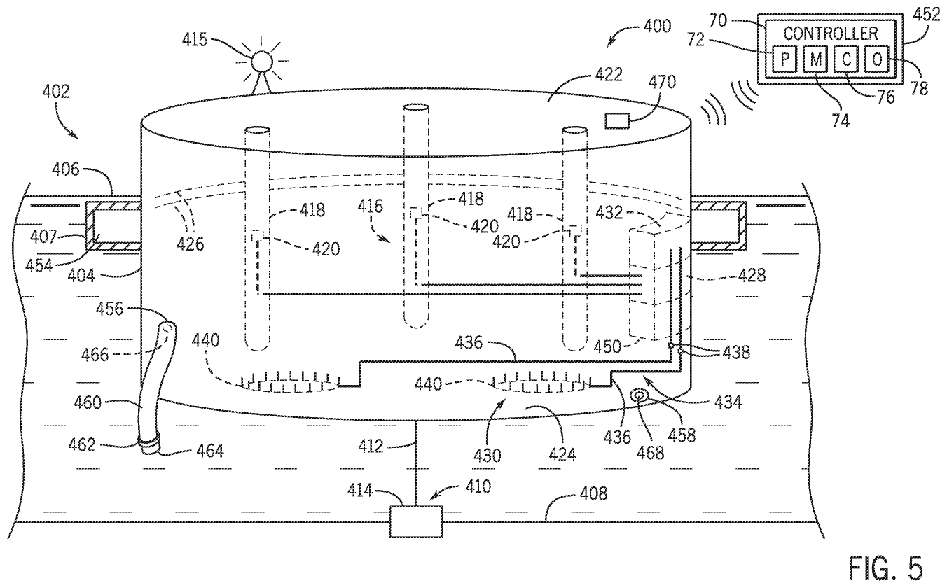

FIG. 5 illustrates an embodiment of a photobioreactor system 400 located at an aquatic environment 402 (e.g., in a body of water). As shown, the photobioreactor system 400 includes a container 404. The container 404 may be supported on and/or form a floating structure that is buoyant to float in water (e.g., at or near a water surface 406 of the body of water). As shown, the container 404 may include or be coupled to a flotation device 407. In some embodiments, the flotation device 407 may be an annular structure that circumferentially surrounds the container 404. Advantageously, the flotation device 407 may also protect the container 404 from external objects (e.g., floating sticks, vessels). The flotation device 407 is shown schematically and in cross-section for image clarity. The container 404 may be buoyant to float in the water such that at least a portion of the container 404 is positioned above the water surface 406, which may facilitate access to the container 404. However, it should be appreciated that other configurations and/or positions in the water are envisioned.

Additionally, as shown, the container 404 may be tethered or anchored to a sea bed 408 (e.g., sediment) via a tether assembly 410, which may include a rope 412 (e.g., cable) and a weight 414 (e.g., anchor; cement block). Further, while the container 404 is shown as tethered or anchored to the sea bed 408 in a relatively shallow body of water (e.g., nearshore) via the tether assembly 410, the photobioreactor system 400 may be kept in place within a relatively deep body of water (e.g., open waters) via the tether assembly 410 (e.g., either by being anchored to the sea bed 408 and/or due to the weight 414 or other structure, such as a sail, being suspended in the water below the container 404). Thus, advantageously, the tether assembly 410 may be or operate as a drift anchor, which may cause objects (e.g., the container 404) at the water surface 406 to follow surface currents instead of wind. The container 404 may include the flotation device 407 and/or the tether assembly 410 to limit and/or to support movement in the body of water, but at least in some embodiments, the container 404 may be devoid of a motor that actively drives movement of the container 404 through the body of water. A light 415 may be mounted to the container 404 to facilitate visualization of the photobioreactor system 400, such as by traveling vessels that may travel in the aquatic environment 402.

The photobioreactor system 400 may include a light system 416, which has at least one transparent light tube 418 that houses at least one respective light source 420. As shown, the light system 416 includes three transparent light tubes 418 that each house at least one respective light source 420; however, the photobioreactor system 400 may include any suitable number of transparent light tubes 418 and/or light sources 420 (e.g., 1, 2, 3, 4, 5, 6, or more) depending on a size of the container 404 and/or other parameters (e.g., appropriate light conditions for desired organisms in the container 404). It should be appreciated that a number of transparent light tubes 418 and/or the light sources 420 may vary and/or be selected based on dimensions (e.g., a length and a width) of the container 404, as well as based on a density of the light sources 420 and/or characteristics (e.g., intensity) of light output by the light sources 420. For example, the number of transparent light tubes 418 may be selected such that the transparent light tubes 418 have a spacing equal to or less than approximately 50 centimeters (cm), 45 cm, 40 cm, 35 cm, 30 cm, 25 cm, or 20 cm, or with the spacing between approximately 20 to 50 cm or 30 to 40 cm. In some embodiments, each transparent light tube 418 may include multiple light sources 420, and the multiple light sources 420 may be distributed about an interior surface of the transparent light tube 418 (e.g., wrapped circumferentially about the interior surface and spaced apart, such as by a few centimeters, between a top end portion and a bottom end portion of the transparent light tube 418). Further, the one or more transparent light tubes 418 may be at any position and/or arranged in any configuration (e.g., form corners of a square configuration). Further, each of the at least one transparent light tubes 418 may be supported within and/or suspended from a surface of the container 404, such as from a lid 422 of the container 404 and/or a bottom surface 424 of the container 404.

Additionally or alternatively, the light system 416 may include one or more light sources 426 that extend along an interior wall of the container 404. For example, the one or more light sources 426 may be arranged along one or more cables (e.g., strings). Thus, the light system 30 may be efficiently applied to the interior wall of the container 404 by wrapping the one or more cables circumferentially about the interior wall and then coupling the one or more cables to the interior wall via fasteners (e.g., welds, threaded bolts, threaded screws, adhesives). While two rows are shown in FIG. 5 for image clarity, it should be appreciated that the one or more light sources 426 may be arranged in discrete rows that are spaced apart (e.g., extend horizontally; spaced apart vertically) between a bottom portion or edge of the interior wall and an upper portion or edge of the interior wall. Further, the light system 416 may include any suitable number of light sources 426 with any suitable characteristics (e.g., emit white light; full spectrum; mimic sunlight). Each light source 420 is coupled to a power source 428.

Additionally, the photobioreactor system 400 may include an air system 430, which has at least one pump 432 and a tubing assembly 434. The tubing assembly 434 includes tubing 436 and at least one backflow preventer 438 (e.g., one per tubing 436), wherein the tubing 436 extends from the at least one pump 432 to a respective air distribution device 440 (e.g., one or more air stones). As shown, the respective air distribution device 440 may include a ring of air flow devices (e.g., multiple air stones) that provide air as well as agitate (e.g., mix) the contents within the container 404. It should be appreciated that the air system 430 may have any of a variety of parts, configurations, and/or features (e.g., bifurcations, weights). The photobioreactor system 400 may include any suitable number of air distribution devices 440 (e.g., 1, 2, 3, 4, 5, 6, or more) depending on a size of the container 404 and/or other parameters.

The photobioreactor system 400 may include a sensor 450, such as a fluorometer that measures phycocyanin, phycoerythrin, and/or chlorophyll within the container 404. The sensor 450 may generate sensor data indicative of growth of desired organisms in the container 404. The photobioreactor system 400 may include any of a variety of sensors, including but not limited to sensors that monitor pH, dissolved oxygen, temperature, and/or pressure within the container 404. As shown, the controller 70 is part of a remote computing system 452 and communication devices (e.g., wireless communication devices) facilitate communication between components (e.g., sensors, lights, pumps) at the container 404 and the remote computing system 452. In this way, the photobioreactor system 400 may enable remote monitoring and/or control of the components at the container 404 via the remote computing system 452. For example, the controller 70 may be configured to receive and to process the sensor data to determine operational parameters for the photobioreactor system 400, to provide recommendations and/or alerts, and so forth. As another example, the operator may provide inputs at the remote computing system 452 to control the operational parameters for the photobioreactor system 400.

Additionally functionality and automation may be provided. For example, the photobioreactor system 400 may include one or more nutrient containers positioned at or nearby the container 404 so as to store, mix, and/or distribute one or more nutrients into the containers 404 and/or the body of water. For example, in FIG. 5, one or more nutrient containers 454 are coupled to and/or supported within the flotation device 407. However, it should be appreciated that the one or more nutrient containers 454 may be at any suitable location (e.g., separately mounted to sides of the container 404). The photobioreactor system 400 may include at least one outlet 456 that is arranged to transfer the contents of the container 404. The outlet 456 may include an opening (e.g., through hole) formed in a wall (e.g., side wall) of the container 404. The outlet 456 may be positioned at or proximate to the water surface 406; however, it should be appreciated that the outlet 456 may be position at any suitable location relative to the water surface 406. The outlet 456 may be coupled to an outflow tube 460, which may include a tube flotation device 462 to maintain an end 464 of the outflow tube 460 at or near the water surface 406 (e.g., at depth equal to or less than approximately 3, 2, 1, 0.5, 0.25, 0.1, or 0.05 meters). Preferably, the outlet 456 is as close to the water surface 406 as possible, such as within 0.05 meters, to increase a likelihood that the contents dispensed from the outlet 456 stay at the water surface 406 for a relatively long duration (e.g., as opposed to if the contents were dispends deeper under the water surface 406). The outflow tube 460 may also be connected to the one or more nutrient containers 454 to distribute additional levels of the one or more nutrients into the body of water along with the contents of the container 404 and/or to separately distribute the one or more nutrients into the body of water (e.g., without the contents of the container 404, such as based on sensor data that indicates insufficient nutrients in the body of water). In some embodiments, the photobioreactor system 400 may include a separate outlet and outflow tube to separately dispense the one or more nutrients directly from the one or more nutrient containers 454 into the body of water. The photobioreactor system 400 may include at least one environmental water inlet 458 that is arranged to transfer water or fluid into the container 404.

The photobioreactor system 400 may include one or more environmental sensors that generate environmental condition sensor data, such as air or water temperature and pressure, wind direction and speed, water current direction and speed, salinity, humidity, turbidity, pH, pCO2, light, oxygen density, dissolved oxygen, conductivity, carbon dioxide, eDNA, anemometer, depth/altitude, nutrients, radiance and irradiance, fisheries echosounder, cameras (IR,VIS), sedimentation, hydrophone array, fluorometer, and so forth.

The controller 70 may be configured to instruct display of the environmental condition sensor data. The controller 70 may be configured to determine that the sensor data indicates that the contents of the container 404 are ready for distribution and that the environmental condition sensor data is favorable to the distribution of the contents of the container 404, and then the controller 70 may instruct a valve 466 to open to distribute the contents of the container 404 from the outlet 456 into the body of water (e.g., with additional mixing from a mixing system at or nearby the container 404). The controller 70 may receive and account for weather conditions, such as wind speed, wave height, and/or surface current strength. The controller 70 may maintain the valve 466 in a closed position to block distribution of the contents of the container 404 from the outlet 456 into the body of water while the wind speed, the wave height, and/or the surface current strength are outside of respective desirable ranges. For example, a threshold wind speed is set to 20 knots, and any wind speed above 20 knots is determined to be a condition to block the distribution, while any wind speed below 20 knots is determined to be a condition to allow the distribution. The controller 70 require that at least two of the following conditions be true to activate the distribution: wind speed below 20 knots, wave height of less than 1.5 meters, surface current of less than 2 knots. Such thresholds may be utilized for offshore conditions (e.g., open ocean), while other thresholds may be utilized for nearshore conditions (e.g., protected for a bay. For example, in nearshore conditions, the thresholds may be set to wind speed below 15 knots, wave height of less than 0.5 meters, and/or surface current of less than 2 knots. In this way, the thresholds may vary based on conditions and/or location. Additionally or alternatively, the controller 70 may activate the distribution based on a time of day to disperse during the day or right at sunrise or just before sunrise to maximize growth and available light. In an embodiment, the air system 50 may distribute based on sensor readings of sufficient ambient light and lack of cloud cover.

Thus, the controller 70 may provide for dynamic, automatic distribution when the desired organisms demonstrate sufficient growth or other parameters that indicate readiness for distribution (e.g., based on sensor data) and during favorable weather conditions. Similarly, the controller 70 may provide block or deactivate the distribution when the desired organisms do not demonstrate sufficient growth or the other parameters that indicate readiness for distribution and/or when the weather conditions are not favorable. Such control actions may be completed automatically without operator input, or the controller 70 may provide a recommendation to the operator via the remote computing system 452 and then instruct the valve 466 to open in response to receipt of confirmation inputs from the operator. The photobioreactor system 400 also includes a valve 468 associated with the at least one environmental water inlet 458. The controller 70 may control the valve 468 to fill the container 404 with water from the body water after the contents of the container 404 are drained via the outlet 456 and/or when the sensor data indicates that additional water from the body of water should be added to the container 404, for example. However, in some cases, the contents are instead extracted from the container 404 by removing the lid 422 and drawing or pumping the contents from an opening that is then exposed at a top of the container 404. Further, additional components (e.g., desired organisms, nutrients, water) may be added the container 404 through the lid 422 or upon removing the lid 422.

The photobioreactor system 400 may be serviced by service vessels that refill organisms, nutrients, and so forth. The photobioreactor system 400 may use solar, wind, and/or battery power as a power source. It should be appreciated that the controller 70 may be located locally at the container 404 (e.g., off-shore), remotely from the container 404 (e.g., on-shore), or the controller 70 may be a distributed controller with components located both locally at the container 404 and remotely from the container 404 to distribute processing to carry out techniques disclosed herein.

In FIG. 5, the power source 428, the air pump 432, and the sensor 450 are positioned within the container 404. In this way, these components are protected from environmental conditions and/or the container 404 may be airtight so as to block entry of undesirable particles and/or exit of the contents, for example. In some embodiments, at least one opening 470 may be provided in the lid 422 and/or other wall of the container 404 for airflow (e.g., CO₂) into and out of the container 404. In such cases, the at least one opening 470 may be covered by a mesh material to block entry of contaminants. Advantageously, with the one or more light sources 420 within the container 404, the container 404 may be formed from an opaque material and/or light characteristics (e.g., intensity) may be more readily adjusted to facilitate growth of the desired organisms within the container 404. However, the container 404 may be formed from a transparent material to utilize sunlight to facilitate growth in combination with a controlled supply of nutrients, agitation, and/or additional light, for example. For example, the container 404 may be formed from plastic, such as a transparent acrylic. Further, the container 404 may be wrapped in a reflective material and/or include interior walls that are formed from the reflective material.

The photobioreactor systems disclosed herein may be part of environmental treatment distribution systems that promote desired environmental treatment effects, such as decreased aquatic hypoxia, increased aquatic oxygenation, increased export of materials to depth (e.g., excess nutrients from rivers, carbon), enhanced fisheries. The desired environmental treatment effects may be created as part of distribution to surface waters of aquatic environments from maritime operating vessels and/or aircraft. In an embodiment, activity is conducted to induce a beneficial change in the physical and/or chemical composition of the aquatic system and/or environment and/or place such as but not limited to carbonate ion content, pH, temperature, salinity, oxygen content, macronutrient concentrations and/or content, micronutrient concentrations and/or content, cation and/or anion concentrations and/or content, dissolved organic compounds, clay contents and/or concentrations, isotopic systematics including that of hydrogen, carbon, oxygen, nitrogen, sulfur, lithium, boron, strontium, calcium, magnesium, lead, or any other element and/or atom and/or molecule or combinations thereof. In an embodiment, plankton and/or other organisms by themselves or in combination with other materials such as but not limited to nutrients such as but not limited to iron, phosphorous, nitrogen, silica, aluminum, manganese, calcium, sodium, potassium, and/or magnesium are grown and held in the photobioreactors and then introduced into aquatic places or environment. In an embodiment, plankton and/or other organisms by themselves or in combination with other materials such as but not limited to nutrients are grown and held in the photobioreactors and then utilized for consumption (e.g., by humans and/or other animals, such as cattle). The photobioreactor systems disclosed herein may include configurations that provide relatively low-cost structures (e.g., the photobioreactor systems 10, 100 of FIGS. 1-3 and/or the photobioreactor system 300 of FIG. 4). Further, the photobioreactor systems disclosed herein may include configurations that facilitate transport from a first location where desired organisms are grown to a second location where the desired organisms are distributed. Further, the photobioreactor systems disclosed herein may facilitate use of containers that float at a water surface of a body of water for controlled growth environment, as well as efficient distribution into the body of water (e.g., the photobioreactor system 400 of FIG. 5; for bioremediation and/or carbon removal). Further, the photobioreactor systems disclosed herein may be used in any of a variety of environments, as part of any of a variety of systems, and so forth. For example, the photobioreactor systems may be utilized in ocean settings, such as for carbon removal and/or bioremediation, and/or in parallel with wastewater treatment systems, such as for bioremediation and/or carbon removal. Further, the photobioreactor systems disclosed herein may include configurations that enable remote monitoring of and/or controlling distribution of the desired organisms from containers of the photobioreactor systems.

While only certain features have been illustrated and described herein, many modifications and changes will occur to those skilled in the art. It is, therefore, to be understood that the appended claims are intended to cover all such modifications and changes as fall within the true spirit of the disclosure. It should be appreciated that the photobioreactor systems disclosed herein may be and/or operate as a bioreactor system (e.g., with light sources turned on or off to encourage growth, depending on desired organism). It should be appreciated that any features shown and/or described with reference to FIGS. 1-5 may be combined in any suitable manner. For example, the photobioreactor systems of FIGS. 1-4 may be supported on and/or form a floating structure (e.g., on a vessel or platform; formed from buoyant material). As another example, the power source, the pump, and/or the sensor(s) may be positioned within the container of the photobioreactor system of FIG. 4. As another example, the light system may include rows of light sources about an interior wall of the container of the photobioreactor system of FIG. 4.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform] ing [a function] . . . ” or “step for [perform] ing [a function] . . . ”, it is intended that such elements are to be interpreted under 35 U.S.C. § 112 (f). However, for any claims containing elements designated in any other manner, it is intended that such elements are not to be interpreted under 35 U.S.C. § 112 (f).

1. A photobioreactor system, comprising:
a frame;
a reflective wall coupled to the frame such that the reflective wall defines and circumferentially surrounds an interior chamber; and
a plurality of light sources coupled to the reflective wall such that the plurality of light sources circumferentially surrounds the interior chamber.

2. The photobioreactor system of claim 1, wherein at least one side opening is defined between a horizontal bar of the frame and an upper edge of the reflective wall to provide access to the interior chamber.

3. The photobioreactor system of claim 1, wherein the frame is an open box structure with a top opening across a top of the frame to provide access to the interior chamber.

4. The photobioreactor system of claim 1, comprising an air system comprising a pump and at least one tubing assembly, wherein the at least one tubing assembly comprises a tubing that extends from the pump to an air stone that is configured to be placed in a container within the interior chamber.

5. The photobioreactor system of claim 1, comprising a controller configured to generate instructions to control the plurality of light sources according to a schedule.

6. The photobioreactor system of claim 5, wherein the controller is configured to receive an input of one or more characteristics of contents in a container within the interior chamber and to select the schedule from a database based on the input.

7. The photobioreactor system of claim 6, wherein the one or more characteristics comprise a type of organism, an intended location of distribution of the contents, or both.

8. The photobioreactor system of claim 1, comprising a container configured to be positioned within the interior chamber, wherein the container is formed from a transparent material.

9. The photobioreactor system of claim 1, wherein the photobioreactor system is disposed on or in a floating or fixed structure at a body of water.

10. The photobioreactor system of claim 1, wherein the frame and the reflective wall coupled to the frame define and circumferentially surround the interior chamber and a plurality of additional interior chambers, and the plurality of light sources coupled to the reflective wall circumferentially surround the interior chamber and the plurality of additional interior chambers.

11. The photobioreactor system of claim 10, wherein the interior chamber and the plurality of additional interior chambers are each configured to receive a respective container that holds and enables growth of a desired organism.

12. A photobioreactor system configured to be positioned proximate to a water surface of a body of water, the photobioreactor system comprising:
a floating structure;
a container that forms the floating structure or that is supported on the floating structure, wherein the container is configured to hold contents that comprise a desired organism;
a plurality of light sources configured to emit light into the container; and
an air system comprising a pump and at least one tubing assembly, wherein the at least one tubing assembly comprises at least one tubing that extends from the pump to at least one air distribution device that is configured to be placed in the container.

13. The photobioreactor system of claim 12, wherein the at least one air distribution device comprises a plurality of air stones.

14. The photobioreactor system of claim 12, comprising a frame assembly that defines an interior chamber configured to receive the container, wherein the frame assembly comprises a frame and at least one reflective wall.

15. The photobioreactor system of claim 12, wherein the plurality of light sources comprise as least one light source that is positioned in the container, discrete rows of light sources positioned about a wall of the container, or both.

16. The photobioreactor system of claim 12, comprising:
a sensor configured to generate sensor data indicative of one or more characteristics of the contents of the container; and
a controller configured to:
receive and process to the sensor data; and
instruct display of a recommendation, an alert, or both on a display screen for visualization by an operator.

17. The photobioreactor system of claim 16, wherein the controller is located on-shore and remotely from the container.

18. The photobioreactor system of claim 16, wherein the controller is configured to control operation of the plurality of light sources and the pump.

19. An environmental treatment method, comprising:
placing a desired organism, nutrients, and water into a container;
placing an air outlet of an air distribution system into the container;
providing a light source to emit light into the container;
monitoring growth of the desired organism within the container;
transferring the container to a distribution site in an aquatic environment; and
distributing contents of the container into the aquatic environment.

20. The environmental treatment method of claim 19, wherein the transferring the container to the distribution site comprises lifting the container from a frame assembly that supports the light source.
